(19) United States
(12) Patent Application Publication
(10) Pub. No.: US 2001/0044420 A1
(43) Pub. Date: Nov. 22, 2001

(54) Combination use of gemcitabine and tumor suppressor gene therapy in the treatment of neoplasms

(76) Inventors: Loretta Lynn Nielsen, Millington, NJ (US); JoAnn Horowitz, Kenilworth, NJ (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(21) Appl. No.: 09/919,145

(22) Filed: Jul. 31, 2001

Related U.S. Application Data
(63) Continuation of application No. 09/522,669, filed on Mar. 10, 2000.
(60) Provisional application No. 60/125,165, filed on Mar. 19, 1999.

(51) Int. Cl. A61K 48/00; A61K 31/7072

(52) U.S. Cl. 514/44; 514/50

(57) ABSTRACT

This invention describes novel methods of treating subjects afflicted with hyperproliferative diseases such as tumors or metastatic disease. In particular, this invention provides methods of treating cancer, comprising the use of gemcitabine in combination with a tumor suppressor gene or gene product. The invention also provides for a pharmacological composition comprising a tumor suppressor protein or a tumor suppressor nucleic acid and gemcitabine, and a kit for the treatment of mammalian cancer or hyperproliferative cells.

### FIELD OF THE INVENTION

This invention describes novel methods of treating subjects afflicted with hyperproliferative diseases such as tumors or metastatic disease. In particular, this invention provides methods of treating cancer, comprising the use of gemcitabine in combination with a tumor suppressor gene or gene product.

### BACKGROUND OF THE INVENTION

Mutation of the p53 gene is the most common genetic alteration in human cancers (Bartek (1991) Oncogene 6: 1699-1703, Hollstein (1991) Science, 253: 49-53), and it has been shown that introduction of wild-type p53 in mammalian cancer cells lacking endogenous wild-type p53 protein suppresses the neoplastic phenotype of those cells (see, e.g., U.S. Pat. No. 5,532,220). International patent publication WO 98/35554 (published Aug. 20, 1998) provides methods of treating hyperproliferative mammalian cells by administering certain adjunctive anti-cancer agents (particularly paclitaxel) in combination with tumor suppressor (e.g., p53) gene therapy to cells that have deficient tumor suppressor activity. International patent publication WO 98/35554 additionally mentions a variety of other chemotherapeutic agents that can also be used in combination with tumor suppressor gene therapy. For example, a tumor suppressor nucleic acid (e.g., a nucleic acid encoding p53) can be administered with an adjunctive anti-cancer agent (e.g., paclitaxel) and a DNA damaging agent such as cisplatin, carboplatin, or navelbine (vinorelbine tartate). However, there is still a need to provide new methods for treating hyperproliferative cells, particularly cancer cells, and thus new methods comprising new combinations of agents would be welcomed, especially in cases where new combinations and/or particular dosage ranges are targeted at specific types of cancers.

### SUMMARY OF THE INVENTION

The present invention provides methods of treating cancer or hyperproliferative mammalian cells comprising contacting said cells with a tumor suppressor protein or tumor suppressor nucleic acid and also contacting said cells with gemcitabine. In a preferred embodiment, the tumor suppressor nucleic acid is a nucleic acid that encodes a wild-type p53 protein.

The cells to be treated are often neoplastic cells, and in particular, a method of treating a cancer is provided. the cancers to be treated include, but are not limited to, an ovarian cancer, pancreatic cancer, a non-small cell lung cancer, small cell lung cancer, primary peritoneal cancer, hepatocarcinoma, melanoma, retinoblastoma, breast tumor, colorectal carcinoma, leukemia, lymphoma, brain tumor, cervical carcinoma, sarcoma, prostate tumor, bladder tumor, tumor of the reticuloendothelial tissues, Wilm's tumor, astrocytoma, glioblastoma, neuroblastoma, ovarian carcinoma, osteosarcoma, renal cancer, or head and neck cancer. In a particularly preferred embodiment, the cancer to be treated is ancreatic cancer.

The tumor suppressor nucleic acid is preferably delivered to the target cell by a vector. Such vectors' viruses have been modified by recombinant DNA technology to enable the expression of the tumor suppressor nucleic acid in the target cell. These vectors may be derived from vectors of non-viral (e.g., plasmids) or viral (e.g., adenovirus, adenoassociated virus, retrovirus, herpes virus, vaccinia virus) origin. In the preferred practice of the invention, the vector is a recombinantly modified adenoviral vector. Non-viral vectors are preferably complexed with agents to facilitate the entry of the DNA across the cellular membrane. Examples of such non-viral vector complexes include the formulation with polycationic agents which facilitate the condensation of the DNA and lipid-based delivery systems. An example of a lipid-based delivery system would include liposome based delivery of nucleic acids.

Particularly suitable adenoviral vectors (e.g., for delivery of a nucleic acid encoding a wild-type p53 protein) comprise a partial or total deletion of a protein IX DNA. In one embodiment, the deletion of the protein IX gene sequence extends from about 3500 bp from the 5′ viral termini to about 4000 bp from the 5′ viral termini. The vector may also comprise a deletion of a non-essential DNA sequence in adenovirus early region 3 and/or in adenovirus early region 4 and in one embodiment the deletion is the DNA sequence E1a and/or E1b. A particularly preferred recombinant adenoviral vector for delivery of a human p53 cDNA comprises the adenovirus type 2 major late promoter or the human CMV promoter, and the adenovirus type 2 tripartite leader cDNA. One such preferred adenoviral vector is ACN53 (also sometimes referred to as “A/C/N/53”) as described, e.g., in WO 95/11984.

The gemcitabine is preferably GEMZAR® and the gemcitabine can be dispersed in a pharmacologically acceptable excipient. The tumor suppressor protein or tumor suppressor nucleic acid and the gemcitabine can both be dispersed in a single composition (comprising one or multiple excipient(s)).

The tumor suppressor (protein or nucleic acid) and/or gemcitabine can be administered intra-arterially, intravenously (e.g., injected), intraperitoneally and/or intratumorally, together or sequentially. Preferred sites of administration include intra hepaticartery, intraperitoneal, or, where it is desired to treat cells in the head (e.g, neurological cells), into the carotid system of arteries.

In a preferred embodiment, the tumor suppressor nucleic acid encodes a wild-type p53 protein and is delivered by a recombinant adenoviral vector administered in a total dose ranging from about 1×10¹⁰ to about 7.5×10¹⁴ adenovirus particles in a treatment regimen selected from the group consisting of: the total dose in a single dose, the total dose divided over 5 days and administered daily, the total dose divided over 15 days and administered daily, the total dose divided over 30 days and administered daily, and the total dose delivered daily for each of five days; and the gemcitabine is administered intravenously in a treatment regimen selected from the group consisting of: a total dose ranging from about 500 to about 1500 mg/m² (preferably 800 to 1000 mg/m²) over a single day on three separate days of a two-week treatment cycle, and a total dose ranging from about 500 to about 1500 mg/m² (preferably 800 to 1000 mg/m²) weekly for up to seven weekly cycles.

Preferred routes of administration include intra-arterial administration (e.g., intra-hepatic artery administration) and intraperitoneal administration.

This invention also provides for kits for the treatment of mammalian cancer or hyperproliferative cells. The kits include a tumor suppressor protein or nucleic acid described herein (more preferably a wild-type p53 protein or nucleic acid (e.g., in a viral or non-viral vector), or a retinoblastoma (RB) protein or nucleic acid) and gemcitabine. The kit can optionally further include instructions describing the administration of both the tumor suppressor protein or nucleic acid and the gemcitabine to treat the cancer or hyperproliferative cells. One particularly preferred kit includes A/C/N/53 and gemcitabine.

In another embodiment this invention provides pharmacological compositions comprising a tumor suppressor protein or a tumor suppressor nucleic acid and gemcitabine.

The cells thus treated include neoplastic cells comprising a cancer selected from the group consisting of an ovarian cancer, mesothelioma, pancreatic cancer, a non-small cell lung cancer, small cell lung cancer, primary peritoneal cancer, hepatocarcinoma, melanoma, retinoblastoma, breast tumor, colorectal carcinoma, leukemia, lymphoma, brain tumor, cervical carcinoma, sarcoma, prostate tumor, bladder tumor, tumor of the reticuloendothelial tissues, Wilm's tumor, astrocytoma, glioblastoma, neuroblastoma, osteosarcoma, renal cancer, and head and neck cancer. The treatment treating preferably results in inhibition of growth or proliferation of a tumor as assayed by measurement of the volume of the tumor.

The invention also provides for a pharmacological composition comprising a tumor suppressor protein or a tumor suppressor nucleic acid and gemcitabine. The tumor suppressor protein or tumor suppressor nucleic acid can be selected from the group consisting of a nucleic acid that encodes a wild-type p53 protein, a nucleic acid that encodes a retinoblastoma (RB) protein, a wild-type p53 protein, and a retinoblastoma (RB) protein.

The nucleic acid can be contained in a recombinant adenoviral vector. The nucleic acid can be contained in a recombinant adenoviral vector comprising a partial or total deletion of a protein IX DNA and comprising a nucleic acid encoding a P53 protein. In one embodiment, the deletion of the protein IX gene sequence can extend from about 3500 bp for the 5′ viral termini to about 4000 bp from the 5′ viral termini. The deletion of DNA can include sequence designated E1a and E1b. The recombinant adenoviral vector can further comprise the adenovirus type 2 major late promoter or the human CMV promoter, the adenovirus type 2 tripartite leader cDNA and a human p53 cDNA. In a preferred embodiment, the vector is A/C/N/53.

The invention provides for a method of treating a metastatic cell, said method comprising contacting said cell with a tumor suppressor nucleic acid or tumor suppressor polypeptide and gemcitabine. The contacting can comprise topical administration of a tumor suppressor nucleic acid to a surgical wound.

### DEFINITIONS

“Tumor suppressor genes” are nucleic acids for which loss-of-function mutations are oncogenic. Thus, the absence, mutation, or disruption of normal expression of a tumor suppressor gene in an otherwise healthy cell increases the likelihood of the cell attaining a neoplastic state. Moreover, when a functional tumor suppressor gene or protein of the present invention is present in a cell, its presence suppresses the tumorigenicity, malignancy or hyperproliferative phenotype of the host cell. Examples of tumor suppressor nucleic acids within this definition include, but are not limited to p110^{RB}, p56^{RB}, p53, and other tumor suppressors described herein and in copending application U.S. Ser. No. 08/328,673 filed on Oct. 25, 1994. Tumor suppressor nucleic acids include tumor suppressor genes, or nucleic acids derived therefrom (e.g., cDNAs, cRNAs, mRNAs, and subsequences thereof encoding active fragments of the respective tumor suppressor polypeptide), as well as vectors comprising these sequences.

A “tumor suppressor polypeptide or protein” refers to a polypeptide that, when present in a cell, reduces the tumorigenicity, malignancy, or hyperproliferative phenotype of the cell.

The term “viral particle” refers to an intact virion. The concentration of infectious adenovirus viral particles is typically determined by spectrophotometric detection of DNA, as described, for instance, by Huyghe (1995) Human Gene Ther. 6:1403-1416.

The terms “neoplasia” or “neoplastic” are intended to describe a cell growing and/or dividing at a rate beyond the normal limitations of growth for that cell type.

The phrase “treating a cell” refers to the inhibition or amelioration of one or more disease characteristics of a diseased cell. When used in reference to a cancer cell that is neoplastic (e.g., a mammalian cancer cell lacking an endogenous wild-type tumor suppressor protein), the phrase “treating a cell” refers to mitigation or elimination of the neoplastic phenotype. Typically such treatment results in inhibition (a reduction or cessation of growth and/or proliferation) of the cell as compared to the same cell under the same conditions but for the treatment. Such inhibition may include cell death (e.g., apoptosis). These terms when used with reference to a tumor refer to inhibition of growth or proliferation of the tumor mass (e.g., as measured volumetrically). Such inhibition may be mediated via reduction in growth rate and/or proliferation rate and/or death of cells comprising the tumor mass. The inhibition of growth or inhibition of proliferation can be accompanied by an alteration in cellular phenotype (e.g., restoration of morphology characteristic of healthy cells, restoration of contact inhibition, loss of invasive phenotype, inhibition of anchorage independent growth, etc.). For the purposes of this disclosure, a diseased cell will have one or more pathological traits. These traits in a diseased cell may include, inter alia, defective expression of one or more tumor suppressor proteins. Defective expression may be characterized by complete loss of one or more functional tumor suppressor proteins or a reduction in the level of expression of one or more functional tumor suppressor proteins. Such cells are often neoplastic and/or tumorigenic.

The term “systemic administration” refers to administration of a composition or drug, such as the recombinant adenoviral vectors of the invention or gemcitabine, in a manner that results in the introduction of the composition or drug into the circulatory system. The term “regional administration” refers to administration of a composition or drug into a specific anatomical space, such as intraperitoneal, intrathecal, subdural, or to a specific organ, and the like. The term “local administration” refers to administration of a composition or drug into a limited, or circumscribed, anatomic space, such as intratumoral injection into a tumor mass, subcutaneous injections, intramuscular injections, and the like. Any one of skill in the art would understand that local administration or regional administration may also result in entry of the composition or drug into the circulatory system.

The term “reduced tumorigenicity” is used herein to refer to the conversion of hyperproliferative (e.g. neoplastic) cells to a less proliferative state. In the case of tumor cells, “reduced tumorigenicity” is intended to mean tumor cells that have become less tumorigenic or non-tumorigenic or non-tumor cells whose ability to convert into tumor cells is reduced or eliminated. Cells with reduced tumorigenicity either form no tumors in vivo or have an extended lag time of weeks to months before the appearance of in vivo tumor growth. Cells with reduced tumorigenicity may also result in slower growing three dimensional tumor mass compared to the same type of cells having fully inactivated or non-functional tumor suppressor gene growing in the same physiological milieu (e.g., tissue, organism age, organism sex, time in menstrual cycle, etc.).

As used herein an “active fragment” of a gene or polypeptide includes smaller portion(s) (subsequences) of the gene or nucleic acid derived therefrom (e.g., cDNA) that retain the ability to encode proteins having tumor suppressing activity. Similarly, an active fragment of a polypeptide refers to a subsequence of a polypeptide that has tumor suppressing protein. One example of an active fragment is p56^{RB} as described, e.g., in copending U.S. Ser. No. 08/328,673 filed on Oct. 25, 1994.

“Nucleic acids”, as used herein, may be DNA or RNA. Nucleic acids may also include modified nucleotides that permit correct read through by a polymerase and do not alter expression of a polypeptide encoded by that nucleic acid.

The phrase “nucleotide sequence” includes both the sense and antisense strands as either individual single strands or in the duplex.

The phrase “DNA sequence” refers to a single or double stranded DNA molecule composed of the nucleotide bases, adenosine, thymidine, cytosine and guanosine.

The phrase “nucleic acid sequence encoding” refers to a nucleic acid which directs the expression of a specific protein or peptide. The nucleic acid sequences include both the DNA strand sequence that is transcribed into RNA and the RNA sequence that is translated into protein. The nucleic acid sequences include both the full length nucleic acid sequences as well as non-full length sequences derived from the full length sequences. It being further understood that the sequence includes the degenerate codons of the native sequence or sequences which may be introduced to provide codon preference in a specific host cell.

The phrase “expression cassette” refers to nucleotide sequences which are capable of affecting expression of a structural gene in hosts compatible with such sequences. Such cassettes include at least promoters and optionally, transcription termination signals. Additional factors necessary or helpful in effecting expression may also be used.

The term “operably linked” as used herein refers to linkage of a promoter upstream from a DNA sequence such that the promoter mediates transcription of the DNA sequence.

“Isolated” or “substantially pure” when referring to nucleic acid sequences encoding tumor suppressor protein or polypeptide or fragments thereof refers to isolated nucleic acids which do not encode proteins or peptides other than the tumor suppressor protein or polypeptide or fragments thereof.

The term “recombinant” refers to DNA which has been isolated from its native or endogenous source and modified either chemically or enzymatically to delete naturally-. occurring flanking nucleotides or provide flanking nucleotides that do not naturally occur. Flanking nucleotides are those nucleotides which are either upstream or downstream from the described sequence or sub-sequence of nucleotides.

A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). It is recognized that vectors often include an expression cassette placing the nucleic acid of interest under the control of a promoter. Vectors include, but are not limited to replicons (e.g., plasmids, bacteriophages) to which fragments of DNA may be attached and become replicated. Vectors thus include, but are not limited to RNA, autonomous self-replicating circular DNA (plasmids), and includes both the expression and nonexpression plasmids. Where a recombinant microorganism or cell culture is described as hosting an “expression vector” this includes both extrachromosomal circular DNA and DNA that has been incorporated into the host chromosome(s). Where a vector is being maintained by a host cell, the vector may either be stably replicated by the cells during mitosis as an autonomous structure, or is incorporated within the host's genome.

The term effective amount is intended to mean the amount of vector or drug which achieves a positive outcome on controlling cell growth and/or proliferation.

The abbreviation “C.I.U.” as used herein, stands for “cellular infectious units.” The C.I.U. is calculated by measuring viral hexon protein positive cells (e.g., −293 cells) after a 48 hr. infection period (Huyghe (1995) Human Gene Ther. 6:1403-1416).

The abbreviation “m.o.i.” as used herein refers to “multiplicity of infection” and is the C.I.U. per cell.

The term “gemcitabine” as used herein refers to the drug commercially known and available as GEMZAR® (Gemcitabine HCI). Gemzar® is a commercially available form of gemcitabine (2′, 2′-difluoro-deoxycytidine, dFdC, Gemzar®), which is a pyrimidine analogue of deoxycytidine in which the deoxyribose moiety contains two fluorine atoms at the 2′-position. (See Heinemann et al. Cancer Res 1988 48:4024). As noted in DeVita et al. (Eds.), Cancer: Principles and Practice of Oncology (Lippencott-Raven, Phila., Pa., 5th Ed. 1997), gemcitabine is known to have a broad spectrum of antitumor activity against leukemias and solid tumors. (reference: Hertel et al., Evaluation of the antitumor activity of gemcitabine (2′, 2′-difluoro-2′deoxycytidine). Cancer Res 1990, 50:4417.) According to DeVita et al., “The most commonly used clinical schedule is a 30-minute IV infusion weekly for 3 weeks followed by a 1-week rest, and the recommended dose is 1000 mg/m². In phase II trials using this schedule (800 to 1250 dFdC mg/m² per week), response rates in the 16% to 24% range were reported in patients with non-small cell lung cancer (previously untreated) and small cell lung cancer, breast cancer patients who had received no more than one prior regimen for metastatic disease, and patients with refractory ovarian cancer, hormone-refractory prostate cancer, and head and neck cancer.” DeVita et al (Eds.), Cancer: Principles and Practice of Oncology (Lippencott-Raven, Phila., Pa., 5th Ed. 1997).

The term “contacting a cell” when referring to contacting with a drug and/or nucleic acid is used herein to refer to contacting in a manner such that the drug and/or nucleic acid is internalized into the cell. In this context, contacting a cell with a nucleic is equivalent to transfecting a cell with a nucleic acid. Where the drug is lipophilic or the nucleic acid is complexed with a lipid (e.g., a cationic lipid) simple contacting will result in transport (active, passive and/or diffusive) into the cell. Alternatively the drug and/or nucleic acid may be itself, or in combination with a carrier composition be actively transported into the cell. Thus, for example, where the nucleic acid is present in an infective vector (e.g., an adenovirus) the vector may mediate uptake of the nucleic acid into the cell. The nucleic acid may be complexed to agents which interact specifically with extracellular receptors to facilitate delivery of the nucleic acid into the cell, examples include ligand/polycation/DNA complexes as described in U.S. Pat. Nos. 5,166,320 and 5,635,383. Additionally, viral delivery may be enhanced by recombinant modification of the knob or fiber domains of the viral genome to incorporate cell targeting moieties.

The constructs designated herein as “A/c/N/53”, “A/M/N/53”, p110^{RB}, p56^{RB}, refer to the constructs so designated in copending application U.S. SER. NO. 08/328,673, filed on Oct. 25, 1994, International Application WO 95/11984. (A/C/N/53 is also sometimes referred to herein as “ACN53”).

A “conservative substitution”, when describing a protein refers to a change in the amino acid composition of the protein that does not substantially alter the protein's activity. Thus, “conservatively modified variations” of a particular amino acid sequence refers to amino acid substitutions of those amino acids that are not critical for protein activity or substitution of amino acids with other amino acids having similar properties (e.g., acidic, basic, positively or negatively charged, polar or non-polar, etc.) such that the substitutions of even critical amino acids do not substantially alter activity. Conservative substitution tables providing functionally similar amino acids are well known in the art. For example, the following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

See also, Creighton (1984) Proteins W. H. Freeman and Company. In addition, individual substitutions, deletions or additions which alter, add or delete a single amino acid or a small percentage of amino acids in an encoded sequence are also “conservatively modified variations”.

### BRIEF DESCRIPTION OF THE DRAWINGS

FIG. 1 shows an isobologram for MiaPaCa2 pancreatic tumor cells treated in vitro with gemcitabine and ACN53 (wild-type p53 gene in an adenoviral vector as described and referenced above). The x-axis indicates the amount of gemcitabine in nM units. The y-axis indicates the amount of ACN53 (p53 Ad) in multiplicity of infection (m.o.i.) units. When the experimental curve falls below the straight diagonal line, as is the case here, this represents a synergistic interaction. (See, e.g., O'Connell, M. A., and Wolfinger, R. D., J. Computational and Graphical Statistics 6: 224-241, 1997, and Berenbaum, M. C., Pharmacol. Rev. 41: 93-141, 1989).

FIG. 2 shows a dose response curve before statistical analysis for of ACN53 (p53 Ad), with dosages expressed as multiplicity of infection (m.o.i.) units along the x-axis, in MiaPaCa2 pancreatic tumor cells, with the y-axis indicating percentage of cell proliferation.

FIG. 3 shows a dose response curve before statistical analysis for gemcitabine (dosages expressed as NM as indicated along the x-axis) in MiaPaCa2 pancreatic tumor cells, with the y-axis indicating percentage of cell proliferation.

FIG. 4 shows a dose response curve before statistical analysis for drug interactions of ACN53 (p53 Ad), with dosages expressed as multiplicity of infection (m.o.i.) units along the x-axis, and gemcitabine (dosages in nM units as indicated in the box to the right of the curves) in MiaPaCa2 pancreatic tumor cells. The y-axis indicates percentage of cell proliferation.

FIG. 5 shows an isobologram for BxPC-3 pancreatic tumor cells treated in vitro with gemcitabine and ACN53 (wild-type p53 gene in an adenoviral vector as described and referenced above). The x-axis indicates the amount of gemcitabine in nM units. The y-axis indicates the amount of ACN53 (p53 Ad) in multiplicity of infection (m.o.i.) units.

FIG. 6 shows a dose response curve before statistical analysis for of ACN53 (p53 Ad), with dosages expressed as multiplicity of infection (m.o.i.) units along the x-axis, in BxPC-3 pancreatic tumor cells, with the y-axis indicating percentage of cell proliferation.

FIG. 7 shows a dose response curve before statistical analysis for gemcitabine (dosages expressed as nM as indicated along the x-axis) in BxPC-3 pancreatic tumor cells, with the y-axis indicating percentage of cell proliferation.

FIG. 8 shows a dose response curve before statistical analysis for drug interactions of ACN53 (p53 Ad), with dosages expressed as multiplicity of infection (m.o.i.) units along the x-axis, and gemcitabine (dosages in nM units as indicated in the box to the right of the curves) in BxPC-3 pancreatic tumor cells. The y-axis indicates percentage of cell proliferation.

FIG. 9 shows an isobologram for MiaPaCa2 pancreatic tumor cells treated in vitro with gemcitabine and ACN53 (wild-type p53 gene in an adenoviral vector as described and referenced above). The x-axis indicates the amount of gemcitabine in nM units. The y-axis indicates the amount of ACN53 (p53 Ad) in multiplicity of infection (m.o.i.) units.

FIG. 10 shows a dose response curve before statistical analysis for of ACN53 (p53 Ad), with dosages expressed as multiplicity of infection (m.o.i.) units along the x-axis, in MiaPaCa2 cells, with the y-axis indicating percentage of cell proliferation.

FIG. 11 shows a dose response curve before statistical analysis for gemcitabine (dosages expressed as nM as indicated along the x-axis) in MiaPaCa2 cells, with the y-axis indicating percentage of cell proliferation.

FIG. 12 shows a dose response curve before statistical analysis for drug interactions of ACN53 (p53 Ad), with dosages expressed as multiplicity of infection (m.o.i.) units along the x-axis, and gemcitabine (dosages in nM units as indicated in the box to the right of the curves) in MiaPaCa2 cells. The y-axis indicates percentage of cell proliferation.

FIG. 13 shows an isobologram for MidT mouse mammary tumor cells treated in vitro with gemcitabine and ACN53 (wild-type p53 gene in an adenoviral vector as described and referenced above). The x-axis indicates the amount of gemcitabine in nM units. The y-axis indicates the amount of ACN53 (p53 Ad) in multiplicity of infection (m.o.i.) units.

FIG. 14 shows a dose response curve before statistical analysis for of ACN53 (p53 Ad), with dosages expressed as multiplicity of infection (m.o.i.) units along the x-axis, in MidT mouse mammary tumor cells, with the y-axis indicating percentage of cell proliferation.

FIG. 15 shows a dose response curve before statistical analysis for gemcitabine (Gemzar®) (dosages expressed as nM as indicated along the x-axis) in MidT mouse mammary tumor cells, with the y-axis indicating percentage of cell proliferation.

FIG. 16 shows a dose response curve before statistical analysis for drug interactions of ACN53 (p53 Ad), with dosages expressed as multiplicity of infection (m.o.i.) units along the x-axis, and gemcitabine (dosages in nM units as indicated in the box to the right of the curves) in MidT mouse mammary tumor cells. The y-axis indicates percentage of cell proliferation.

### DETAILED DESCRIPTION

The present invention provides new methods of treating cancer or hyperproliferative mammalian cells. The methods comprise contacting the cells with a tumor suppressor protein or tumor suppressor nucleic acid and also contacting the cells with gemcitabine. Typically, the tumor suppressor protein or nucleic acid used will be the same species as the tumor suppressor protein that is lacking. Thus, where the cell lacks endogenous p53 activity, a p53 protein or p53 nucleic acid will be used. More information on p53 gene therapy can be found, e.g., in International patent publication WO 98/35554 (published Aug. 20, 1998), which is expressly incorporated herein by reference.

In still another embodiment, this invention provides for advantageous treatment regimens utilizing gemcitabine in combination with tumor suppressor genes or gene products.

The tumor suppressor protein or nucleic acid can be administered in a single dose or a multiplicity of treatments, e.g., each separated by at least about 6 hours, more preferably in least three treatments separated by about 24 hours.

In a preferred embodiment, the tumor suppressor nucleic acid encodes a wild-type p53 protein and is delivered by a recombinant adenoviral vector administered in a total dose ranging from about 1×10¹⁰ to about 7.5×10¹⁴ adenovirus particles in a treatment regimen selected from the group consisting of: the total dose in a single dose, the total dose divided over 5 days and administered daily, the total dose divided over 15 days and administered daily, the total dose divided over 30 days and administered daily, and the total dose delivered daily for each of five days; and the gemcitabine is administered intravenously in a treatment regimen selected from the group consisting of: a total dose ranging from about 500 to about 1500 mg/m² (preferably 800 to 1000 mg/m²) over a single day on three separate days of a two-week treatment cycle, and a total dose ranging from about 500 to about 1500 mg/m² (preferably 800 to 1000 mg/m²) weekly for up to seven weekly cycles.

In another preferred embodiment in cases where the gemcitabine is administered in a total dose ranging from about 500 to about 1500 mg/m² over a single day on three separate days of a two-week treatment cycle, the method is repeated every 28 days to achieve two or more cycles (preferably six cycles), the two-week treatment cycles for gemcitabine being spaced apart by a two week non-dosing rest period (during which preferably no gemcitabine is administered).

In a preferred embodiment in cases where the gemcitabine is administered in a total dose ranging from about 500 to about 1500 mg/m² over a single day on three separate days of a two-week treatment cycle, the gemcitabine is preferably administered on days 1, 7, and 14 of the two-week treatment cycle. Thus, by way of example, during a second cycle under this particular regimen the gemcitabine is preferably administered on days 29, 35, and 42 (counting from the first day of the first cycle). This regimen is preferably carried out for six cycles.

In another preferred embodiment, the gemcitabine is administered in a total dose of about 1000 mg/m² weekly for seven weekly cycles.

In another preferred embodiment, the tumor suppressor nucleic acid encoding p53 is delivered by a recombinant adenoviral vector and administered in a total (undivided) daily dose ranging from about 7.5×10¹¹ to about 7.5×10¹³ adenovirus particles. (Preferably, the total daily dose is administered for each of five days, preferably for six cycles, with a non-dosing period of about 14 to 23 days between each dosing period of a cycle).

In another preferred embodiment, the tumor suppressor nucleic acid encoding p53 is delivered by a recombinant adenoviral vector administered in a total (undivided) daily dose of about 7.5×10¹³ adenovirus particles, and the gemcitabine is administered intravenously in a total dose of about 800 mg/m² over a single day on three separate days of a two-week treatment cycle.

The order in which the tumor suppressor and gemcitabine are administered is not critical to the invention. Thus the composition(s) can be administered simultaneously or sequentially.

Preferred routes of administration include intra-arterial administration (e.g., intra-hepatic artery administration) and intraperitoneal administration.

Routes of Delivery

Pharmaceutical compositions can be delivered by any means known in the art, e.g., systemically, regionally, or locally; by intraarterial, intratumoral, intravenous (IV), parenteral, intra-pleural cavity, topical, oral, or local administration, as subcutaneous, intratracheal (e.g., by aerosol) or transmucosal (e.g., buccal, bladder, vaginal, uterine, rectal, nasal mucosa), intra-tumoral (e.g., transdermal application or local injection). Particularly preferred modes of administration include intra-arterial injections, especially when it is desired to have a “regional effect,” e.g., to focus on a specific organ (e.g., pancreas, brain, liver, spleen, lungs). For example, intra-hepatic artery injection is preferred if the anti-tumor regional effect is desired in the liver; or, an intra-arterial injection can be for an anti-tumor regional effect in the pancreas; or, intra-carotid artery injection can be used where it is desired to deliver a composition to the brain, (e.g., for treatment of brain tumors, administration can be into a carotid artery or an artery of the carotid system of arteries (e.g., occipital artery, auricular artery, temporal artery, cerebral artery, maxillary artery, etc).

The pharmaceutical compositions of this invention are also useful for parenteral administration, such as intravenous administration or administration into a body cavity or lumen of an organ. The pharmaceutical compositions of this invention are also useful for peritoneal administration into the peritoneal cavity, e.g., to treat ovarian cancer.

Treatment Regimens

The pharmaceutical compositions can be administered in a variety of unit dosage forms depending upon the method of administration. Dosages for gemcitabine are well known to those of skill in the art. Moreover, such dosages are typically advisorial in nature and may be adjusted depending on the particular therapeutic context, patient tolerance, etc. Actual methods for preparing parenterally administrable compositions will be known or apparent to those skilled in the art. Detailed information for preparing pharmaceutical compositions can be found in such publications as Remington's Pharmaceutical Science, 15th ed., Mack Publishing Company, Easton, Pa.

The compositions containing the active ingredients can be administered for therapeutic treatments. In therapeutic applications, compositions are administered to a patient suffering from a disease characterized by cancer or hyperproliferation of one or more cell types in an amount sufficient to cure or at least partially arrest the disease and/or its complications. An amount adequate to accomplish this is defined as a “therapeutically effective dose.” Amounts effective for this use will depend upon the severity of the disease and the general state of the patient's health.

Single or multiple administrations of the compositions may be administered depending on the dosage and frequency as required and tolerated by the patient. In any event, the composition should provide a sufficient quantity of the active ingredients of this invention to effectively treat the patient.

Tumor Suppressor Genes and Gene Products

As indicated above, a p53 tumor suppressor gene is particularly preferred for use in the methods of this invention. Methods of cloning p53 into vectors suitable for expression of the respective tumor suppressor proteins or for gene therapy applications are well known to those of skill in the art. Thus, for example, the cloning and use of p53 is described in detail by Wills (1994) supra; in U.S. Pat. No. 5,532,220, in copending U.S. Ser. No. 08/328,673 filed on Oct. 25, 1994, and in WO 95/11984. Typically the expression cassette is constructed with the tumor suppressor cDNA operably linked to a promoter, more preferably to a strong promoter (e.g., the Ad2 major late promoter (Ad2 MLP), or the human cytomegalovirus immediate early gene promoter (CMV)). In a particularly preferred embodiment, the promoter is followed by the tripartite leader cDNA and the tumor suppressor cDNA is followed by a polyadenylation site (e.g., the E1b polyadenylation site) (see, e.g., copending U.S. Ser. No. 08/328,673, WO 95/11984 and Wills (1994) supra). It will be appreciated that various tissue-specific promoters are also suitable. Thus, for example, a tyrosinase promoter can be used to target expression to melanomas (see, e.g., Siders (1996) Cancer Res. 56:5638-5646). In a particularly preferred embodiment, the tumor suppressor cDNA is expressed in a vector suitable for gene therapy as described herein.

One of skill will appreciate that many conservative variations of the nucleic acid and polypeptide sequences described herein yield functionally identical products. For example, due to the degeneracy of the genetic code, “silent substitutions” (i.e., substitutions of a nucleic acid sequence which do not result in an alteration in an encoded polypeptide) are an implied feature of every nucleic acid sequence which encodes an amino acid. Similarly, “conservative amino acid substitutions,” in one or a few amino acids in an amino acid sequence are substituted with different amino acids with highly similar properties (see, the definitions section, supra), are also readily identified as being highly similar to a disclosed amino acid sequence, or to a disclosed nucleic acid sequence which encodes an amino acid. Such conservatively substituted variations of each explicitly described sequence are a feature of the present invention.

One of skill would recognize that modifications can be made to the tumor suppressor proteins without diminishing their biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly His) placed on either terminus to create conveniently located restriction sites or termination codons or purification sequences.

Modifications to nucleic acids and polypeptides may be evaluated by routine screening techniques in suitable assays for the desired characteristic. For instance, changes in the immunological character of a polypeptide can be detected by an appropriate immunological assay. Modifications of other properties such as nucleic acid hybridization to a target nucleic acid, redox or thermal stability of a protein, hydrophobicity, susceptibility to proteolysis, or the tendency to aggregate are all assayed according to standard techniques.

The tumor suppressors used in the methods of this invention can be introduced to the cells either as a protein or as a nucleic acid. Where the tumor suppressor is provided as a protein, a tumor suppressor gene expression product (e.g., a p53 or an RB polypeptide or fragment thereof possessing tumor suppressor activity) is delivered to the target cell using standard methods for protein delivery (see discussion, below). Alternatively, where the tumor suppressor is a tumor suppressor nucleic acid (e.g., a gene, a cDNA, an MRNA, etc.) the nucleic acid is introduced into the cell using conventional methods of delivering nucleic acids to cells. These methods typically involve delivery methods of in vivo or ex vivo gene therapy as described below. Particularly preferred methods of delivering p53 or RB include lipid or liposome delivery and/or the use of retroviral or adenoviral vectors.

In Vivo Gene Therapy

In a more preferred embodiment, the tumor suppressor nucleic acids (e.g., cDNA(s) encoding the tumor suppressor protein) are cloned into gene therapy vectors that are competent to transfect cells (such as human or other mammalian cells) in vitro and/or in vivo.

Several approaches for introducing nucleic acids into cells in vivo, ex vivo and in vitro have been used. For a review of gene therapy procedures, see, e.g., Zhang (1996) Cancer Metastasis Rev. 15:385-401; Anderson, Science (1992) 256: 808-813; Nabel (1993) TIBTECH 11: 211-217; Mitani (1993) TIBTECH 11: 162-166; Mulligan (1993) Science, 926-932; Dillon (1993) TIBTECH 11: 167-175; Miller (1992) Nature 357: 455-460; Van Brunt (1988) Biotechnology 6(10): 1149-1154; Vigne (1995) Restorative Neurology and Neuroscience 8: 35-36; Kremer (1995) British Medical Bulletin 51(1) 31-44; Haddada (1995) in Current Topics in Microbiology and Immunology, Doerfler and Bohm (eds) Springer-Verlag, Heidelberg Germany; and Yu (1994) Gene Therapy, 1:13-26.

The vectors useful in the practice of the present invention are typically derived from viral genomes. Vectors which may be employed include recombinantly modified enveloped or non-enveloped DNA and RNA viruses, preferably selected from baculoviridiae, parvoviridiae, picomoviridiae, herpesveridiae, poxviridae, adenoviridiae, or picornnaviridiae. Chimeric vectors may also be employed which exploit advantageous ments of each of the parent vector properties (See e.g., Feng (1997) Nature Biotechnology 15:866-870. Such viral genomes may be modified by recombinant DNA techniques to include the tumor suppressor gene and may be engineered to be replication deficient, conditionally replicating or replication competent. In a preferred practice of the invention, the vectors are replication deficient or conditionally replicating. Preferred vectors are derived from the adenoviral, adeno-associated viral and retroviral genomes.

Conditionally replicating viral vectors are used to achieve selective expression in particular cell types while avoiding untoward broad spectrum infection. Examples of conditionally replicating vectors are described in Bischoff, et al.(1996) Science 274:373-376; Pennisi, E. (1996) Science 274:342-343; Russell, S. J. (1994) Eur. J. of Cancer 30A(8):1165-1171. Additionally, the viral genome may be modified to include inducible promoters which achieve replication or expression of the transgene only under certain conditions. Examples of inducible promoters are known in the scientific literature (See, e.g. Yoshida and Hamada (1997) Biochem. Biophys. Res. Comm. 230:426-430; Iida, et al. (1996) J. Virol. 70(9):6054-6059; Hwang, et al.(1997) J. Virol 71(9):7128-7131; Lee, et al. (1997) Mol. Cell. Biol. 17(9):5097-5105; and Dreher, et al.(1997) J. Biol. Chem 272(46); 29364-29371. The transgene may also be under control of a tissue specific promoter region allowing expression of the transgene only in particular cell types.

In a particularly preferred embodiment, the tumor suppressor gene is expressed in an adenoviral vector suitable for gene therapy. The use of adenoviral vectors in vivo, and for gene therapy, is well described in the patent and scientific literature, e.g., see, Hermens (1997) J. Neurosci. Methods., January, 71(1): 85-98; Zeiger (1996) Surgery 120:921-925; Channon (1996) Cardiovasc Res. 32:962-972; Huang (1996) Gene Ther. 3:980-987; Zepeda (1996) Gene Ther. 3:973-979; Yang (1996) Hum. Mol. Genet. 5:1703-1712; Caruso (1996) Proc. Natl. Acad. Sci. USA 93:11302-11306; Rothmann (1996) Gene Ther. 3:919-926; Haecker (1996) Hum. Gene Ther. 7:1907-1914. The use of adenoviral vectors is described in detail in WO 96/25507. Particularly preferred adenoviral vectors are described by Wills (1994) supra; in copending U.S. Ser. No. 08/328,673, and WO 95/11984.

Particularly preferred adenoviral vectors include a deletion of some or all of the protein IX gene. In one embodiment, the adenoviral vectors include deletions of the E1a and/or E1b sequences. In a most preferred embodiment, the adenoviral construct is a p53 encoding construct such as AIC/N/53 or A/M/N/53 (see, e.g., U.S. Ser. No. 08/328,673, and WO 95/11984).

Also preferred are vectors derived from the human adenovirus type 2 or type 5. Such vectors are preferably are replication deficient by modifications or deletions in the E1a and/or E1b coding regions. Other modifications to the viral genome to achieve particular expression characteristics or permit repeat administration or lower immune response are preferred. More preferred are recombinant adenoviral vectors having complete or partial deletions of the E4 coding region, optionally retaining E4 ORF6 and ORF 6/7. The E3 coding sequence may be deleted but is preferably retained. In particular, it is preferred that the promoter operator region of E3 be modified to increase expression of E3 to achieve a more favorable immunological profile for the therapeutic vectors. Most preferred are human adenoviral type 5 vectors containing a DNA sequence encoding p53 under control of the cytomegalovirus promoter region and the tripartite leader sequence having E3 under control of the CMV promoter and deletion of E4 coding regions while retaining E4 ORF6 and ORF 6/7. In the most preferred practice of the invention as exemplified herein, the vector is ACN53.

In a particularly preferred embodiment, the tumor suppressor gene is p53 or RB. As explained above. the cloning and use of p53 is described in detail by Wills (1994) supra; in copending U.S. Ser. No. 08/328,673 filed on Oct. 25, 1994, and in WO 95/11984.

Ex Vivo Gene Therapy

Ex vivo application of the methods of this invention, in particular, provide means for depleting a suitable sample of pathologic hyperproliferative cells. Thus, for example hyperproliferative cells contaminating hematopoietic precursors during bone marrow reconstitution can be eliminated by the ex vivo application of the methods of this invention. Typically such methods involve obtaining a sample from the subject organism. The sample is typically a heterogenous cell preparation containing both phenotypically normal and pathogenic (hyperproliferative) cells. The sample is contacted with the tumor suppressor nucleic acids or proteins and the gemcitabine according to the methods of this invention. The tumor suppressor gene can be delivered, e.g., in a viral vector, such as a retroviral vector or an adenoviral vector. The treatment reduces the proliferation of the pathogenic cells thereby providing a sample containing a higher ratio of normal to pathogenic cells which can be reintroduced into the subject organism.

Ex vivo cell transformation for diagnostics, research, or for gene therapy (e.g., via re-infusion of the transformed cells into the host organism) is well known to those of skill in the art. In a preferred embodiment, cells are isolated from the subject organism, transfected with the tumor suppressor gene or cDNA of this invention, and re-infused back into the subject organism (e.g., patient). Various cell types suitable for ex vivo transformation are well known to those of skill in the art. Particular preferred cells are progenitor or stem cells (see, e.g., Freshney (1994) Culture of Animal Cells, a Manual of Basic Technique, third edition Wiley-Liss, New York, and the references cited therein for a discussion of how to isolate and culture cells from patients). Transformed cells are cultured by means well known in the art. See, also Kuchler (1977) Biochemical Methods in Cell Culture and Virology, Kuchler, R. J., Dowden, Hutchinson and Ross, Inc., and Atlas (1993) CRC Handbook of Microbiological Media (Parks ed) CRC press, Boca Raton, Fla. Mammalian cell systems often will be in the form of monolayers of cells, although mammalian cell suspensions are also used. Alternatively, cells can be derived from those stored in a cell bank (e.g., a blood bank). Illustrative examples of mammalian cell lines include the HEC-1-B cell line, VERO and Hela cells, Chinese hamster ovary (CHO) cell lines, W138, BHK, Cos-7 or MDCK cell lines (see, e.g., Freshney, supra).

Formulations

Pharmaceutically acceptable carriers are determined in part by the particular composition being administered, as well as by the particular method used to administer the composition. Accordingly, there is a wide variety of suitable formulations of pharmaceutical compositions of the present invention. Formulations suitable for oral administration of pharmaceutical compositions comprising the tumor suppressor-expressing nucleic acids can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, tragacanth, microcrystalline cellulose, acacia, gelatin, colloidal silicon dioxide, croscarmellose sodium, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, usually sucrose and acacia or tragacanth, as well as pastilles comprising the active ingredient in an inert base, such as gelatin and glycerin or sucrose and acacia emulsions, gels, and the like containing, in addition to the active ingredient, carriers known in the art.

The packaged nucleic acids, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the packaged nucleic acid with a base, including, for example, liquid triglycerides, polyethylene glycols, and paraffin hydrocarbons.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration and intravenous administration are the preferred methods of administration. The formulations of packaged nucleic acid can be presented in unit-dose or multi-dose sealed containers, such as ampules and vials.

Formulations of the invention as injection solutions and suspensions can be prepared from sterile powders, granules, and tablets of the kind previously described. The exact composition of the formulation, the concentration of the reagents and nucleic acid in the formulation, its pH, buffers, and other parameters will vary depending on the mode and site of administration (e.g., whether systemic, regional or local administration) and needs related to storage, handling, shipping, and shelf life of the particular pharmaceutical composition. Optimization of these parameters depending on the particular need of the formulation can be done by routine methods; and any of ingredients and parameters for known injectable formulations can be used. One example of a suitable formulation is, e.g., a recombinant wild type p53-expressing adenovirus vector of the invention (ACN53) at a concentration of about 7.5×10¹⁰ to 7.5×10¹⁰ particles per ml, sodium phosphate monohydrate at 0.42 mg/ml, sodium phosphate dibasic anhydride at 2.48 mg/ml, sodium chloride at sodium phosphate monohydrate at 5.8 mg/ml, sucrose at 20.0 mg/ml, magnesium chloride hexahydrate at 0.40 mg/ml, typically stored in 1.0 ml dosages. An exemplary formulation for enhanced stability during storage and distribution, especially at refrigeration temperatures, uses rAd5/p53 (at also about 7.5×10¹¹ to 7.5×10¹⁰ particles per ml), sodium phosphate monobasic dihydrate at 1.7 mg/ml, tromethamine (Trizma, or, Tris base, Sigma Chemical Co., St. Louis, Mo.) at 1.7 mg/ml, magnesium chloride hexahydrate at 0.4 mg/ml, sucrose at 20 mg/ml, glycerol at 100 mg/ml, typically stored in 1.0 ml dosages. (See, e.g., U.S. patnet application U.S. Ser. No. 09/249,646 filed Feb. 12, 1999, expressly incorporated herein by reference). Polysorbate 80 at 0.15 mg/ml may optionally be added to the aforesaid exemplary formulation.

The use of delivery-enhancing agents is described in detail in copending U.S. patent application U.S. Ser. No. 08/889,335 filed on Jul. 8, 1997, and International Application Publication No. WO 97/25072, Jul. 17, 1997, and in U.S. patent application U.S. Ser. No. 09/112,074 filed on Jul. 8, 1998, International Application PCT/US 98/14241.

Cells transduced by the packaged nucleic acid as described above in the context of ex vivo therapy can also be administered intravenously or parenterally as described above.

The dose administered to a patient, in the context of the present invention, should be sufficient to effect a beneficial therapeutic response in the patient over time. The dose will be determined by the efficacy of the particular vector employed and the condition of the patient, as well as the body weight or surface area of the patient to be treated. The size of the dose also will be determined by the existence, nature, and extent of any adverse side-effects that accompany the administration of a particular vector, or transduced cell type in a particular patient.

In determining the effective amount of the vector to be administered in the treatment, the physician evaluates circulating plasma levels of the vector, vector toxicities, progression of the disease, and the production of anti-vector antibodies. The typical dose for a nucleic acid is highly dependent on route of administration and gene delivery system. Depending on delivery method the dosage can easily range from about 1 ìg to 100 mg or more. In general, the dose equivalent of a naked nucleic acid from a vector is from about 1 ìg to 100 ìg for a typical 70 kilogram patient, and doses of vectors which include a viral particle are calculated to yield an equivalent amount of therapeutic nucleic acid.

For administration, transduced cells of the present invention can be administered at a rate determined by the LD₅₀ of the vector, or transduced cell type, and the side-effects of the vector or cell type at various concentrations, as applied to the mass and overall health of the patient. Administration can be accomplished via single or divided doses as described below.

In a preferred embodiment, prior to infusion, blood samples are obtained and saved for analysis. Vital signs and oxygen saturation by pulse oximetry are closely monitored. Blood samples are preferably obtained 5 minutes and 1 hour following infusion and saved for subsequent analysis. In ex vivo therapy, leukopheresis, transduction and reinfusion can be repeated are repeated, e.g., every 2 to 3 months. After the first treatment, infusions can be performed on a outpatient basis at the discretion of the clinician. If the reinfusion is given as an outpatient, the participant is monitored for at least 4, and preferably 8 hours following the therapy.

As described above, the adenoviral constructs can be administered systemically (e.g., intravenously), regionally (e.g., intraperitoneally) or locally (e.g., intra- or peri-tumoral or intracystic injection, e.g., to treat bladder cancer). Particularly preferred modes of administration include intra-arterial injection, e.g., in the treatment of pancreatic cancer, or intra-hepatic artery injection for treatment of cancers or tumors of the liver; or, where it is desired to deliver a composition to a brain tumor, a carotid artery or an artery of the carotid system of arteries (e.g., occipital artery, auricular artery, temporal artery, cerebral artery, maxillary artery, etc.). Delivery for treatment of lung cancer can be accomplished, e.g., by use of a bronchoscope. Typically such administration is in an aqueous pharmacologically acceptable buffer as described above. However, in other embodiments, the adenoviral constructs or the tumor suppressor expression cassettes are administered in a lipid formulation, more particularly either complexed with liposomes to for lipid/nucleic acid complexes (e.g., as described by Debs and Zhu (1993) WO 93/24640; Mannino (1988) supra; Rose, U.S. Pat. No. 5,279,833; Brigham (1991) WO 91/06309; and Felgner (1987) supra) or encapsulated in liposomes, more preferably in immunoliposomes directed to specific tumor markers. It will be appreciated that such lipid formulations can also be administered topically, systemically, or delivered via aerosol.

Administration of Tumor Suppressor Proteins

Tumor suppressor proteins (polypeptides) can be delivered directly to the tumor site by injection, or administered locally, regionally, or systemically as described above. In a preferred embodiment, the tumor suppressor proteins are combined with a pharmaceutically acceptable carrier (excipient) to form a pharmacological composition as described above. The tumor suppressor polypeptide will be administered in a therapeutically effective dose. Thus the compositions will be administered in an amount sufficient to cure or at least partially arrest the disease and/or its complications. Amounts effective for this use will depend upon the severity of the disease and the general state of the patient's health.

It will be recognized that tumor suppressor polypeptides, when administered orally, must be protected from digestion. This is typically accomplished either by complexing the polypeptide with a composition to render it resistant to acidic and enzymatic hydrolysis or by packaging the polypeptide in an appropriately resistant carrier such as a liposome as described above. Means of protecting polypeptides for oral delivery are well known in the art (see, e.g., U.S. Pat. No. 5,391,377 describing lipid compositions for oral delivery of therapeutic agents).

Combination Pharmaceuticals

The tumor suppressor nucleic acid or polypeptide can be administered before the gemcitabine (tumor suppressor pretreatment) or the gemcitabine can be administered before the tumor suppressor nucleic acid and/or polypeptide. Of course the tumor suppressor nucleic acid and/or polypeptide and the gemcitabine can be administered simultaneously.

In one embodiment, the tumor suppressor nucleic acid and/or polypeptide and the gemcitabine are administered as a single pharmacological composition. In this embodiment, the tumor suppressor nucleic acid and/or polypeptide and gemcitabine can be suspended or solubilized in a single homogeneous delivery vehicle. Alternatively the tumor suppressor nucleic acid and/or polypeptide and the gemcitabine can each be suspended or solubilized in different delivery vehicles which in turn are suspended (disbursed) in single excipient either at the time of administration or continuously. Thus, for example, gemcitabine may be solubilized in a solvent and the tumor suppressor nucleic acid may be complexed with a lipid which are then either stored together in a suspension or, alternatively are combined at the time of administration. Various suitable delivery vehicles, excipients, etc., are described above.

Therapeutic Kits

In another embodiment, this invention provides for therapeutic kits. The kits include, but are not limited to, a tumor suppressor nucleic acid or polypeptide or a pharmaceutical composition thereof. The kits also include gemcitabine or a pharmaceutical composition thereof or pharmaceutical composition thereof. The various compositions may be provided in separate containers for individual administration or for combination before administration. Alternatively the various compositions may be provided in a single container. The kits may also include various devices, buffers, assay reagents and the like for practice of the methods of this invention. In addition, the kits may contain instructional materials teaching the use of the kit in the various methods of this invention (e.g., in the treatment of cancer, tumors, in the prophylaxis and/or treatment of metastases, and the like).

### EXAMPLES

The following examples are offered to illustrate, but not to limit the claimed invention. The gemcitabine dosing solution in the examples below was made by dissolving 32 mg lyophilized Gemzar® powder (Ely Lilly) in 0.5 ml DMSO to make 100 mM Gemcitabine stock. Appropriate volumes of the stock solution were added to the wells of cells+culture media to give the desired concentrations of Gemcitabine.

### Example A

ACN53 (p53 Ad) Synergizes with Gemcitabine to Inhibit the Proliferation of MiaPaCa2 Pancreatic Tumor Cells

Methods:

Cells: MiaPaCa2 pancreatic tumor cells (p53^{mut}).

ACN53 (p53 Ad) and gemcitabine were both added to cells on day 0. Cell death was quantitated on day 3 using the MTT assay of Mosmann (see Mosmann, T. (1983) J. Immunol. Meth., 65: 55-63). The data was analyzed using the Thin Plate Spline methodology of O'Connell and Wolfinger (1997) (See O'Connell, M. A., and Wolfinger, R. D., J. Computational and Graphical Statistics 6: 224-241, 1997).

Results:

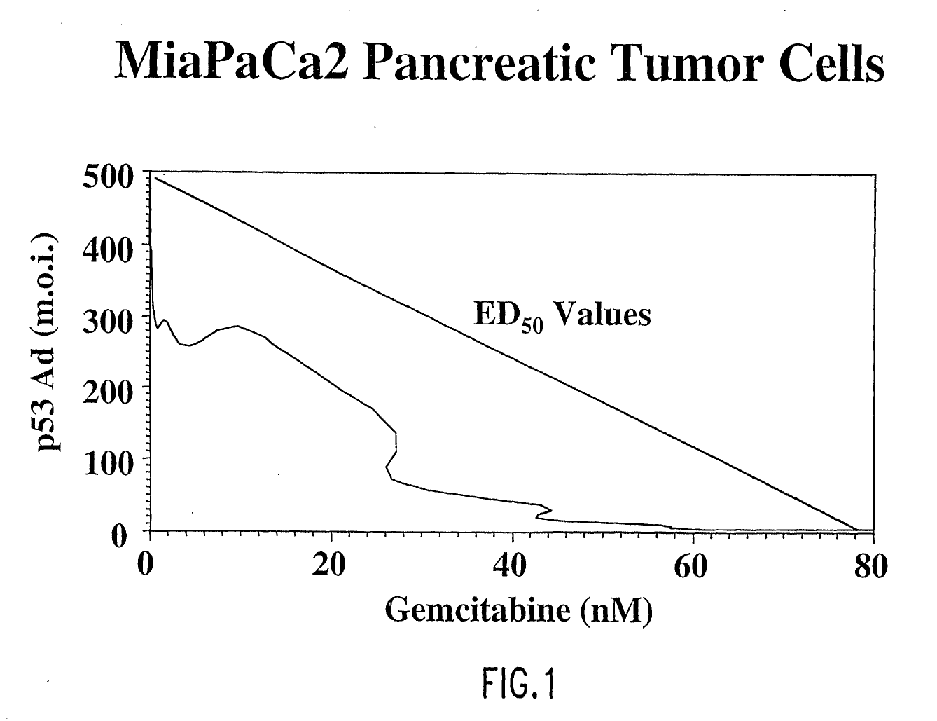

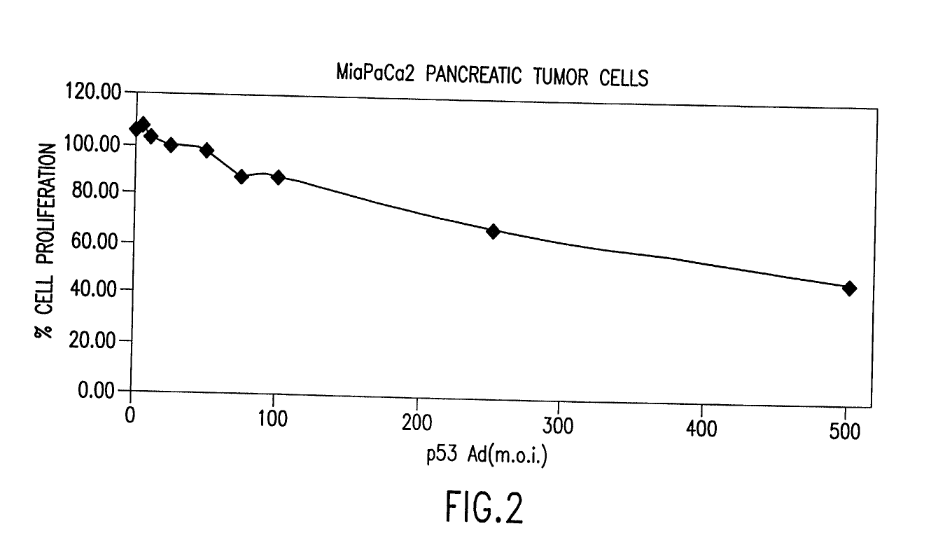

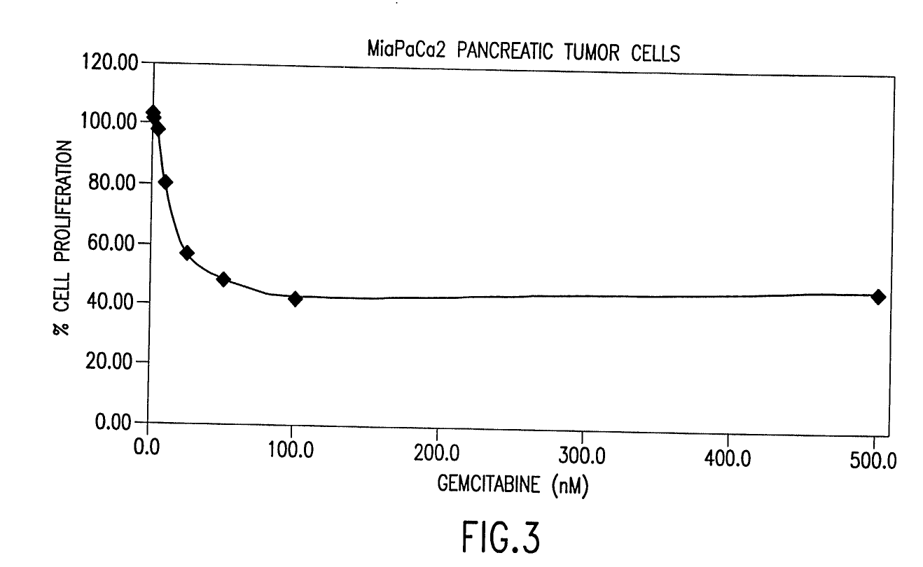

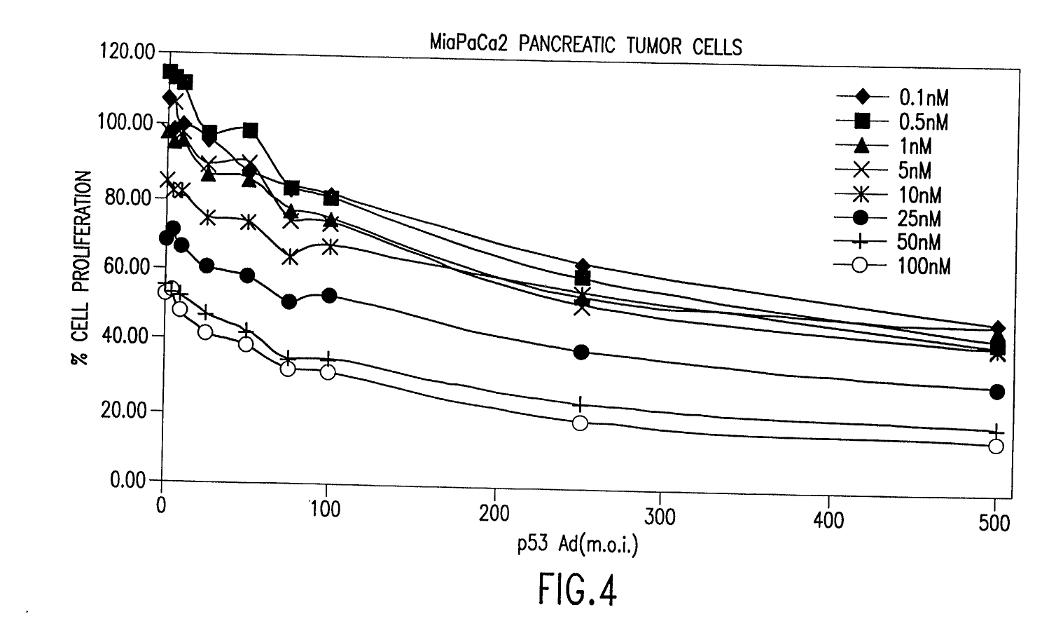

ACN53 (p53 Ad) and gemcitabine had synergistic efficacy (p=0.0001). FIG. 1 shows the isobologram analysis for the interaction of these two agents. FIGS. 2, 3 and 4 show the dose response curves before statistical analysis.

### Example B

ACN53 (p53 Ad) Has Additive Efficacy with Gemcitabine Against the Proliferation of BxPC-3 Pancreatic Tumor Cells

Methods:

Cells: BxPC-3 pancreatic tumor cells (p53^{mut}, K-ras^{mut}).

ACN53 (p53 Ad) and gemcitabine were both added to cells on day 0. Cell death was quantitated on day 3 using the MTT assay. The date was analyzed using the Thin Plate Spline methodologty of O'Connell and Wolfinger (1997).

Results:

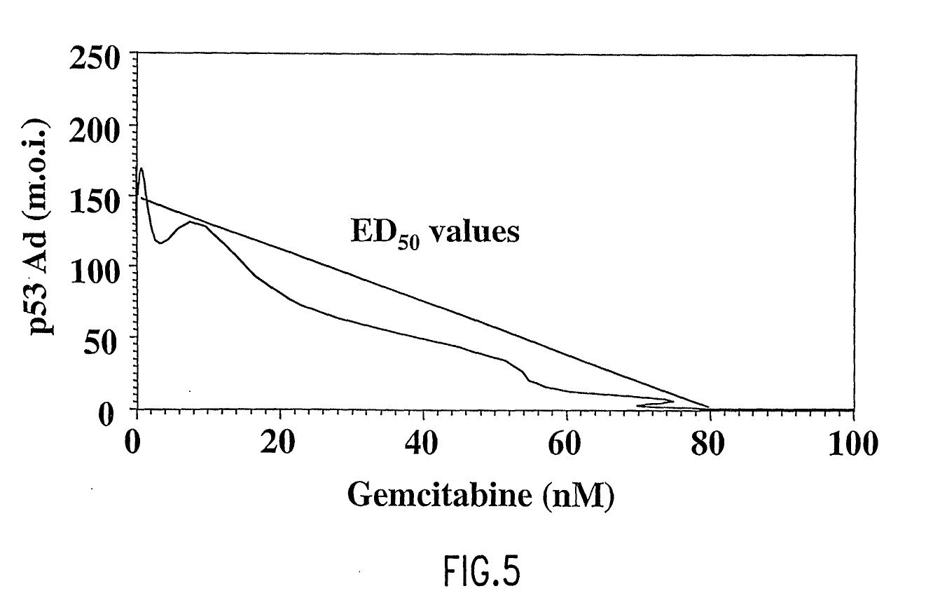

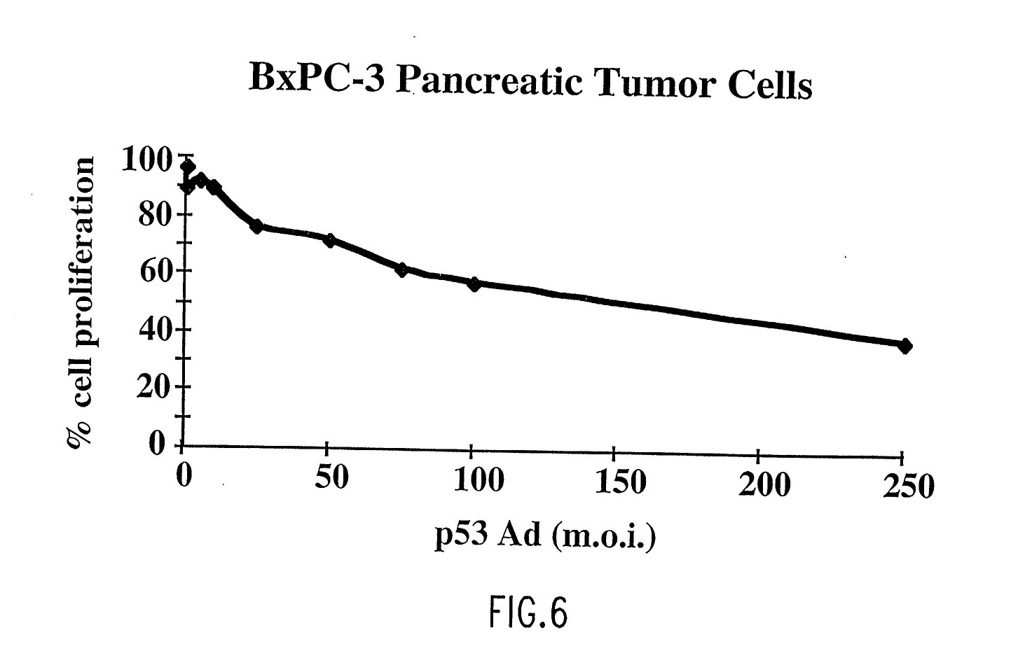

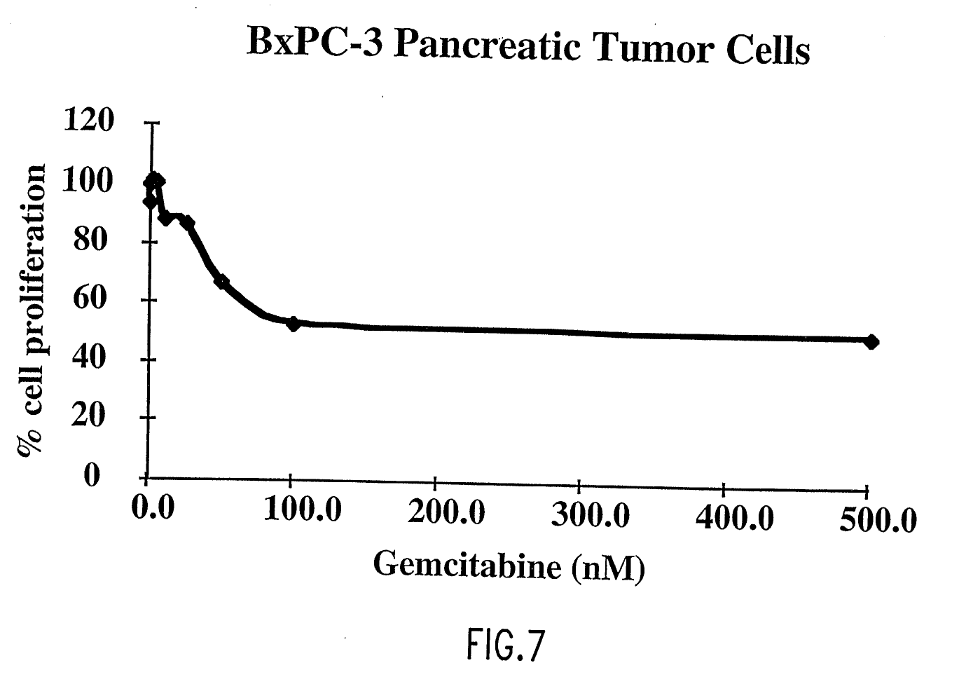

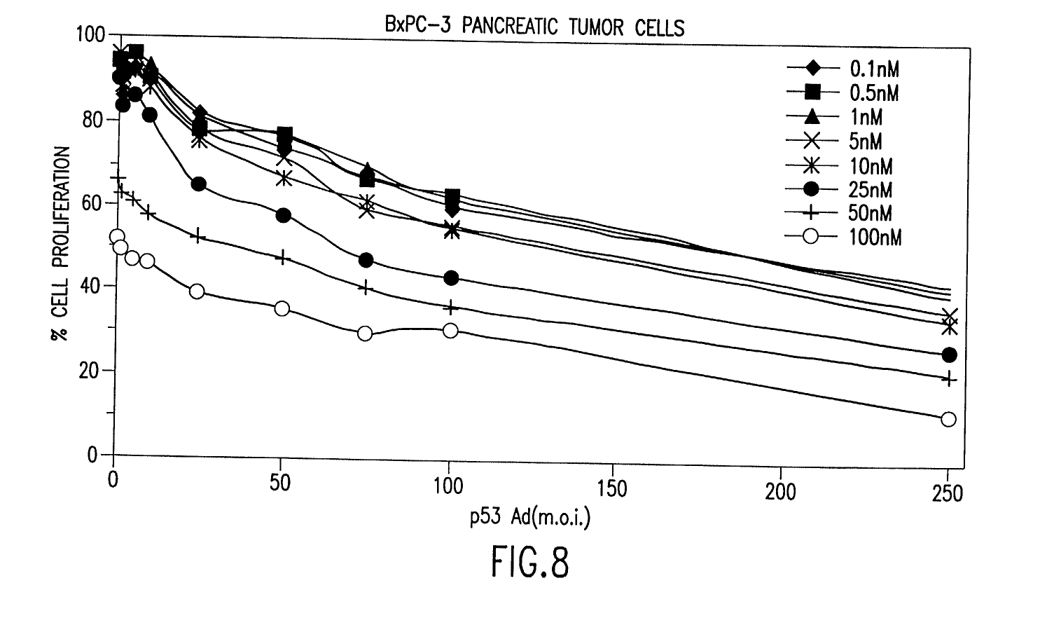

ACN53 (p53 Ad) and gemcitabine had additive efficacy (p=0.6239). FIG. 5 shows the isobologram analysis for the interaction of these two agents. FIGS. 6, 7 and 8 show the dose response curves before statistical analysis.

### Example C

ACN53 (p53 Ad) Synergizes with Gemcitabine to Inhibit the Proliferation of MiaPaCa2 Pancreatic Tumor Cells

Methods:

Cells: MiaPaCa2 pancreatic tumor cells (p53^{mut}, K-ras^{mut}).

Gemcitabine was added to cells on day 0 and ACN53 (p53 Ad) was added on day 1. Cell death was quantitated on day 4 using the MTT assay. The data analyzed using the Thin Plate Spline methodology of O'Connell and Wolfinger (1997).

Results:

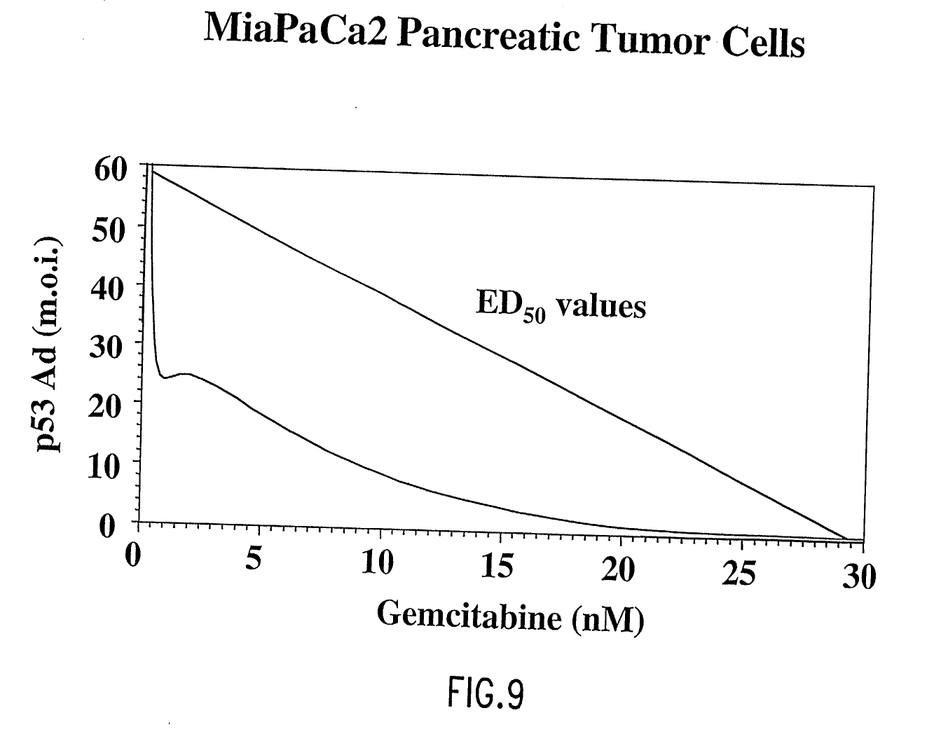

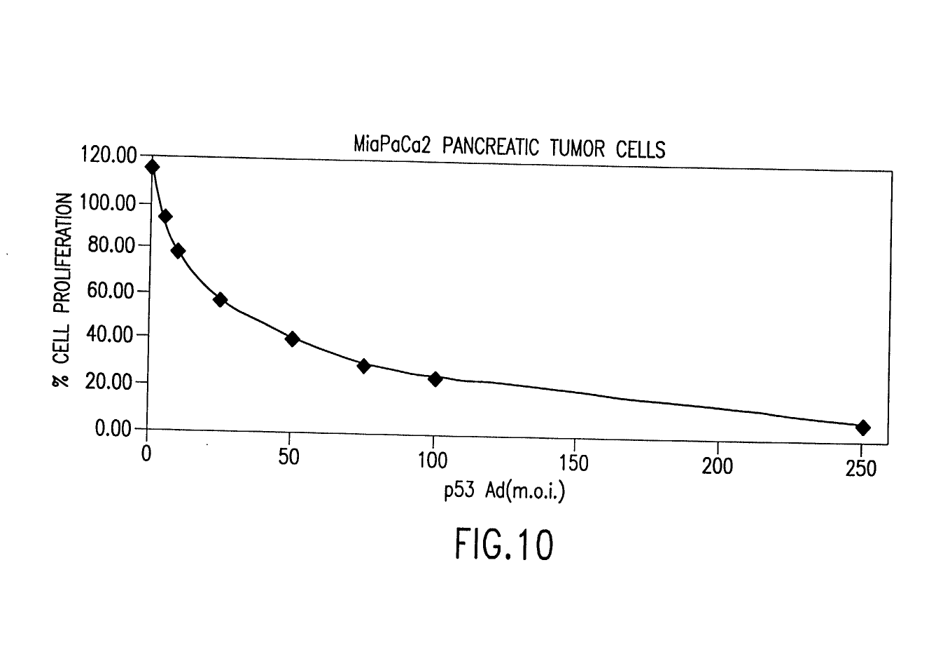

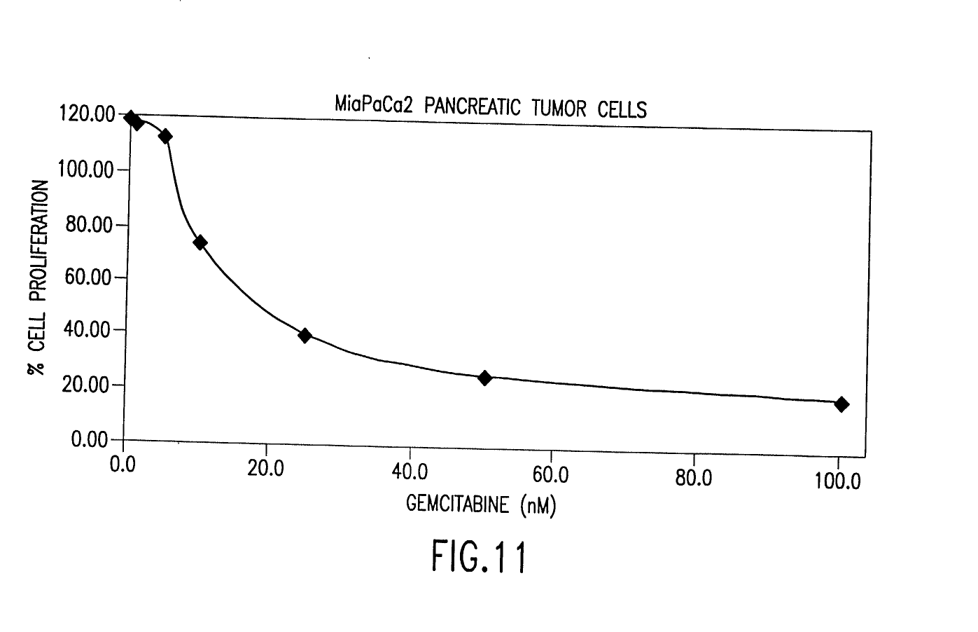

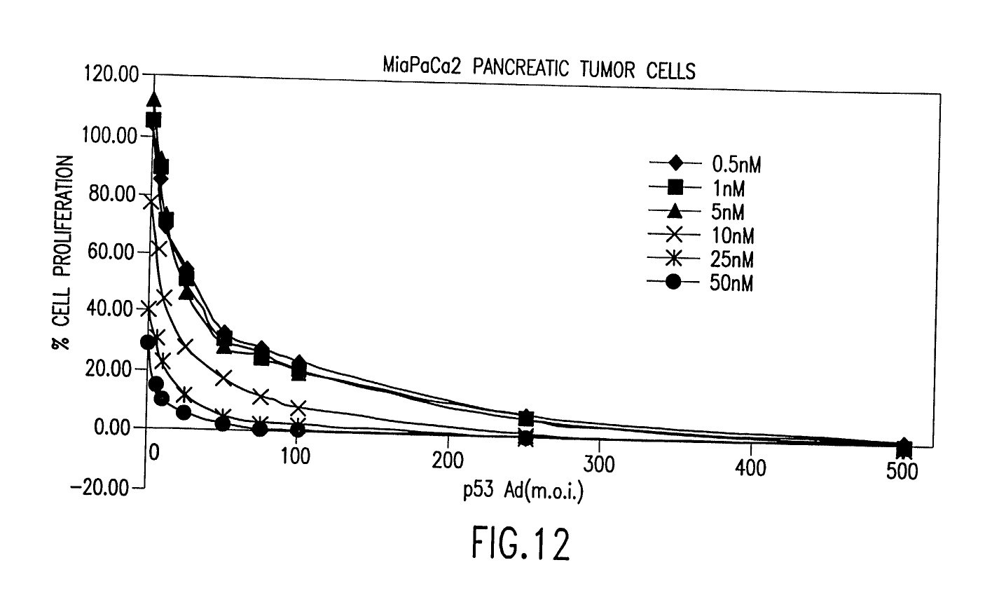

ACN53 (p53 Ad) and gemcitabine had synergistic efficacy (p=0.0106). FIG. 9 shows the isobologram analysis for the interaction of these two agents. FIGS. 10, 11 and 12 show the dose response curves before statistical analysis.

### Example D

Efficacy of SCH58500 and Gemcitabine Against the Proliferation of Midt Mammary Tumor Cells

Methods:

Cells: MidT mouse mammary tumor cells.

ACN53 (p53 Ad) and gemcitabine were both added to cells on day 0. Cell death was quantitated on day 3 using the MTT assay. The data was analyzed using the Thin Plate Spline methodology of O'Connell and Wolfinger (1997).

Results:

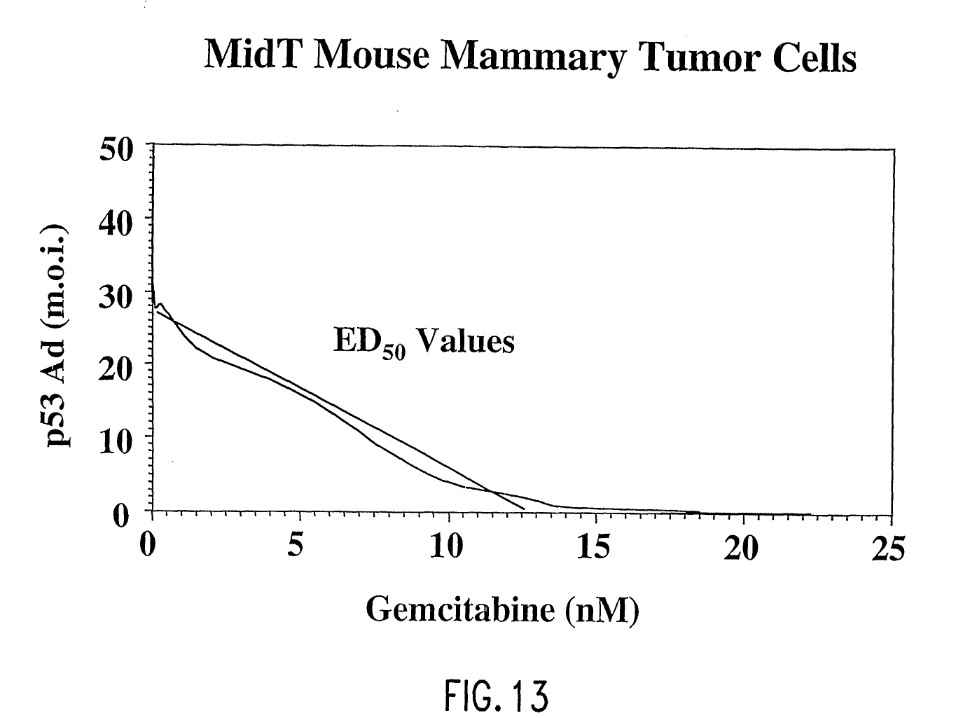

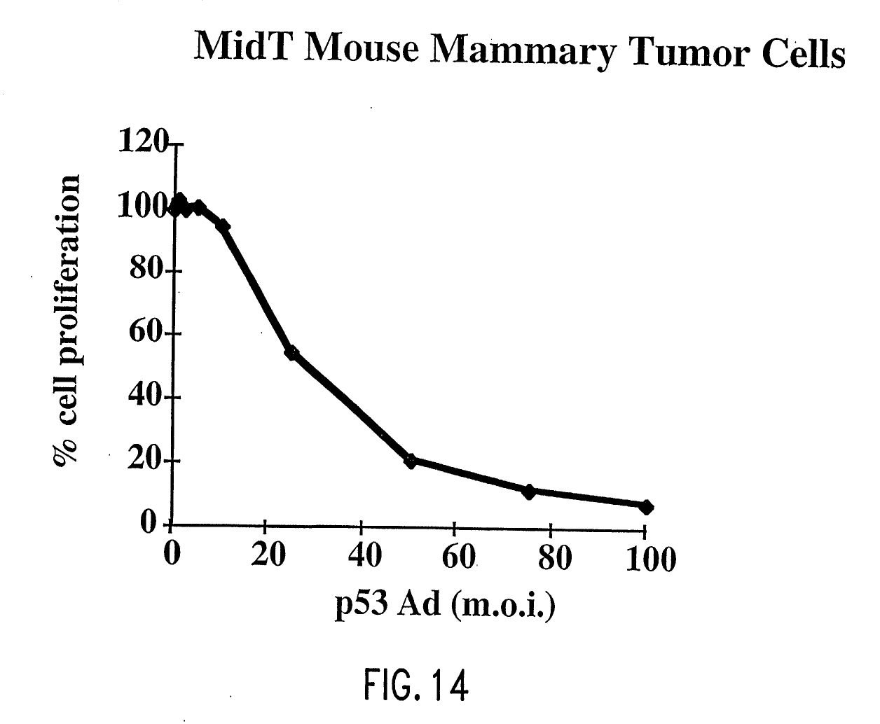

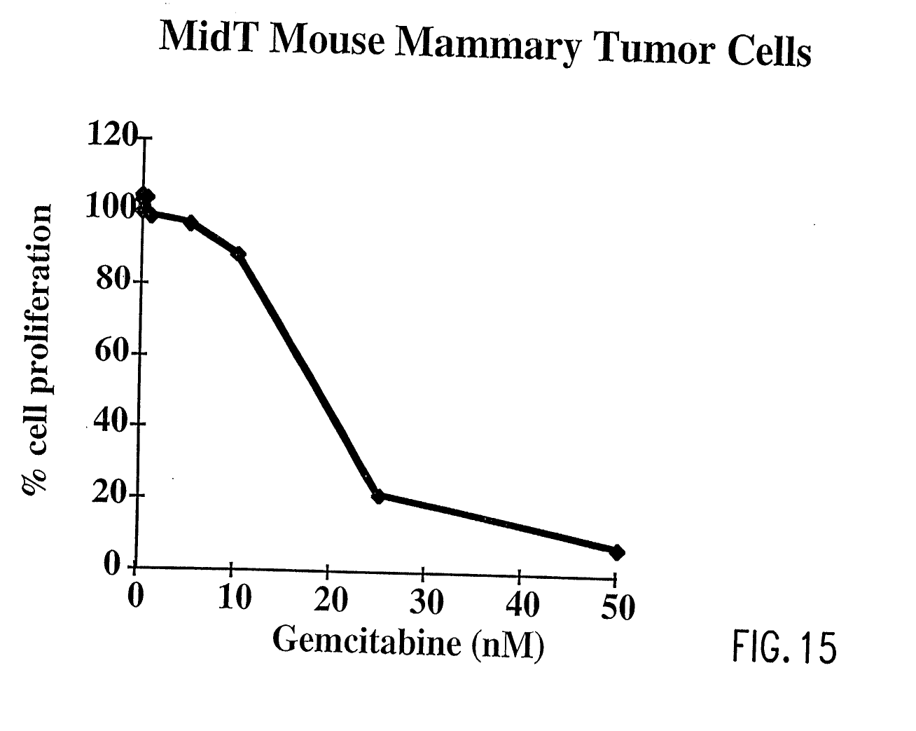

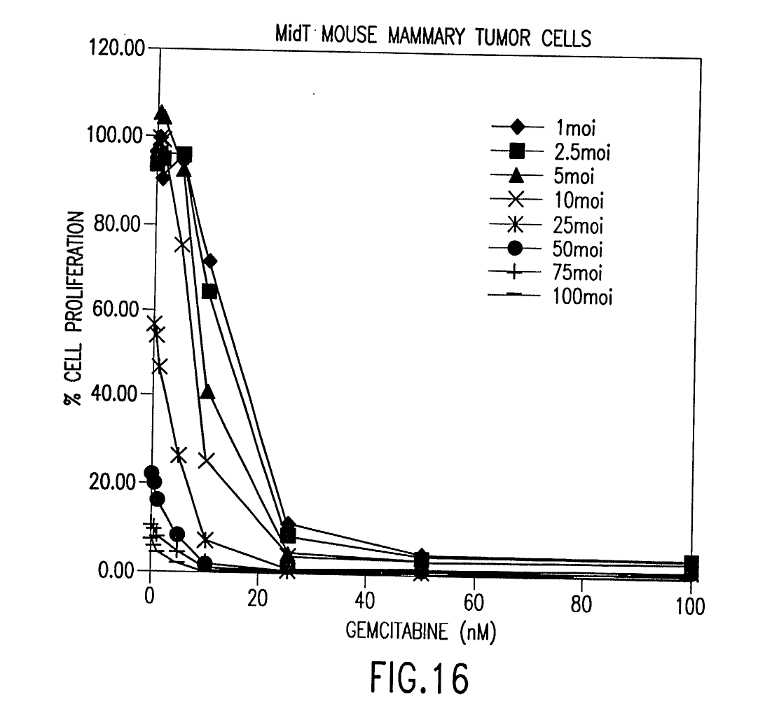

ACN53 and gemcitabine had additive efficacy (p=0.7308 for synergy). FIG. 13 shows the isobologram analysis for the interaction of these two agents. FIGS. 14, 15 and 16 show the dose response curves before statistical analysis.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference.

What is claimed is:

1. A method of treating mammalian cancer or hyperproliferative cells, said method comprising contacting said cells with a tumor suppressor protein or tumor suppressor nucleic acid and also contacting said cells with gemcitabine.

2. The method of claim 1, wherein said tumor suppressor nucleic acid is a nucleic acid that encodes a tumor suppressor protein selected from the group consisting of a wild-type p53 protein and a retinoblastoma (RB) protein.

3. The method of claim 2, wherein said tumor suppressor nucleic acid encodes a wild-type p53 protein.

4. The method of claim 1, wherein said nucleic acid is delivered by a vector selected from the group consisting of a naked DNA plasmid, a plasmid within a liposome, a plasmid complexed with a lipid, a viral vector, an AAV vector, and a recombinant adenoviral vector.

5. The method of claim 1, wherein said nucleic acid is delivered by a recombinant adenoviral vector.

6. The method of claim 5, wherein said vector is A/C/N/53.

7. The method of claim 1, wherein said cells are first contacted with said tumor suppressor nucleic acid or tumor suppressor protein and is subsequently contacted with gemcitabine.

8. The method of claim 1, wherein said cells are first contacted with gemcitabine and subsequently contacted with said tumor suppressor protein or tumor suppressor nucleic acid.

9. The method of claim 1, wherein said cells are simultaneously contacted with gemcitabine and with said tumor suppressor protein or tumor suppressor nucleic acid.

10. The method of claim 1, wherein said cells are cancer cells selected from the group consisting of ovarian cancer, pancreatic cancer, a non-small cell lung cancer, small cell lung cancer, primary peritoneal cancer, hepatocarcinoma, melanoma, retinoblastoma, breast tumor, colorectal carcinoma, leukemia, lymphoma, brain cancer, cervical carcinoma, sarcoma, prostate tumor, bladder cancer, cancer of the reticuloendothelial tissues, Wilm's tumor, astrocytoma, glioblastoma, neuroblastoma, osteosarcoma, renal cancer, and head and neck cancer.

11. The method of claim 1, wherein said contacting comprises intra-arterial injection of the tumor suppressor protein or tumor suppressor nucleic acid.

12. The method of claim 1, wherein said contacting comprises intra-arterial injection of a tumor suppressor nucleic acid for the treatment of pancreatic cancer.

13. The method of claim 1, wherein said contacting comprises intraperitoneal administration of the tumor suppressor protein or tumor suppressor nucleic acid for the treatment of ovarian cancer.

14. The method of claim 1, wherein said contacting comprises injecting the tumor suppressor proteinor tumor suppressor nucleic acid into a tumor.

15. The method of claim 14, wherein said contacting comprises injecting the tumor suppressor protein or tumor suppressor nucleic acid into a tumor for the treatment of lung cancer.

16. The method of claim 1, wherein said contacting comprises injecting gemcitabine into a tumor.

17. The method of claim 1, wherein said contacting comprises intravenously injecting the gemcitabine.

18. The method of claim 1, comprising contacting said cells with A/C/N/53 and gemcitabine.

19. The method of claim 3, wherein:
the tumor suppressor nucleic acid encoding a wild-type p53 protein is delivered by a recombinant adenoviral vector and is administered in a total dose ranging from about 1×10¹⁰ to about 7.5×10¹⁴ adenovirus particles in a treatment regimen selected from the group consisting of: the total dose in a single dose, the total dose divided over 5 days and administered daily, the total dose divided over 15 days and administered daily, the total dose divided over 30 days and administered daily, and the total dose delivered daily for each of five days; and
the gemcitabine is administered intravenously in a treatment regimen selected from the group consisting of: a total dose ranging from about 500 to about 1500 mg/m² over a single day on three separate days of a two-week treatment cycle, and a total dose ranging from about 500 to about 1500 mg/m² weekly for up to seven weekly cycles.

20. The method of claim 19, wherein the gemcitabine is administered in a total dose ranging from about 500 to about 1500 mg/m² over a single day on three separate days of a two-week treatment cycle; and the method is repeated every 28 days to achieve two or more cycles, the two-week treatment cycles for gemcitabine being spaced apart by a two week rest period.

21. The method of claim 20, wherein said method is repeated for six cycles.

22. The method of claim 19, wherein the gemcitabine is administered on day 1, day 7, and day 14 of the two-week treatment cycle.

23. The method of claim 19, wherein the gemcitabine is administered in a total dose of about 1000 mg/M² weekly for seven weekly cycles.

24. The method of claim 19, wherein the tumor suppressor nucleic acid is administered in a total daily dose ranging from about 7.5×10¹¹ to about 7.5×10¹³ adenovirus particles.

25. The method of claim 19, wherein the tumor suppressor nucleic acid is administered in a total dose of about 7.5×10¹³ adenovirus particles, and the gemcitabine is administered intravenously in a total daily dose of about 800 mg/m² over a single day on three separate days of a two-week treatment cycle.

26. A kit for the treatment of mammalian cancer or hyperproliferative cells, said kit comprising:
a first container comprising a tumor suppressor protein or nucleic acid selected from the group consisting of wild-type p53 protein or nucleic acid, or a retinoblastoma (RB) protein or nucleic acid; and
a second container comprising gemcitabine.

27. The kit of claim 26, wherein said tumor suppressor nucleic acid encodes a wild-type p53 protein.

28. The kit of claim 26, further comprising instructions describing the administration of both the tumor suppressor protein or nucleic acid and the gemcitabine to treat cancer or to inhibit the growth or proliferation of said cell.

29. The kit of claim 26, wherein said first container contains a nucleic acid that is contained in a recombinant adenoviral vector.

30. The kit of claim 29, wherein said vector is A/C/N/53.

31. A composition comprising a mammalian cancer or hyperproliferative cell, wherein said cell contains an exogenous a tumor suppressor nucleic acid or a tumor suppressor protein and gemcitabine.

32. The composition of claim 31, wherein said tumor suppressor nucleic acid encodes a wild-type p53 protein.

33. The composition of claim 31, wherein said cells are present in a mammal.

34. A method of treating cancer in a patient in need of such treatment, said treatment comprising administering gemcitabine in combination with a tumor suppressor protein or tumor suppressor nucleic acid.

35. The method of claim 34, wherein the cancer is selected from the group consisting of ovarian cancer, pancreatic cancer, non-small cell lung cancer, small cell lung cancer, primary peritoneal cancer, hepatocarcinoma, melanoma, retinoblastoma, breast tumor, colorectal carcinoma, leukemia, lymphoma, brain tumor, cervical carcinoma, sarcoma, prostate tumor, bladder tumor, tumor of the reticuloendothelial tissues, Wilm's tumor, astrocytoma, glioblastoma, neuroblastoma, osteosarcoma, renal cancer, and head and neck cancer.
